(19) United States
(12) Patent Application Publication
(10) Pub. No.: US 2009/0311293 A1
(43) Pub. Date: Dec. 17, 2009

(54) Method and composition for obtaining odor-suppressing textile products and textile products, namely garments, thus obtained

(76) Inventors: Marcello Fratini, Monte Argentario(Grosseto) (IT); Paolo Salinaro, Sesto Fiorentino (Firenze) (IT)

Correspondence Address: POLLACK, P.C., THE CHRYSLER BUILDING, 132 EAST 43RD STREET, SUITE 760, NEW YORK, NY, 10017, US

(21) Appl. No.: 11/919,144

(22) PCT Filed: Apr. 21, 2005
(86) PCT No.: PCT/IT2006/000237

§ 371 Date: Oct. 21, 2007

(51) Int. Cl. A61K 8/02; A61Q 15/00; A61K 8/26

(52) U.S. Cl. 424/401

(57) ABSTRACT

A method of obtaining a textile product having odor-suppressing properties. The product is treated with a composition which comprises, a polymeric binder, in addition to an active ingredient of aluminum chloride, for stably fixing the active ingredient to the product and releasing it gradually over the course of time. The polymeric binder preferably comprises at least one resin selected from a group including: acrylic-based resin, silicone-based resin, butadiene-based resin, polyurethane-based resin, polyamide-based resin and acrylonitrile-based resin.

### FIELD OF THE INVENTION

The present invention relates to a method for obtaining garments, and more generally textile products, capable of suppressing the formation of the bad smell caused by sweating. It therefore extends, also to textile products obtained with the method.

### DESCRIPTION OF THE PRIOR ART

It is well known that human body can emanate bad smell due to sweating. This phenomenon is even more perceptible when, especially with hot weather, the conditions are favorable for the birth and the development of bacteria that decompose the body secretions, transform them in bad-smelling substances. In fact, it is common experience that the sweat is practically odor-free as soon as it is produced by the body, and that the longer the body remains in contact with the sweat and with the garments impregnated with it, the more unpleasant is the odor developed.

For suppressing the development of the bacteria that cause the bad odor, the use of aluminium salts, namely aluminium chloride, as a deodorizing agent. Various ways of applying said deodorizing agent directly to a fabric have also been proposed, in order to obtain a garment that is intrinsically provided with odor-suppressing properties (see for instance the published PCT international patent application n. WO02/49591).

However, these proposals have not resulted satisfactory, basically due to two kinds of problems: the impregnation of the textile fiber with the deodorizing agent changes the physical characteristics of the fiber itself, with consequent worsening of its quality; the association of the deodorizing agent to the textile fiber is not sufficiently stable, thereby the odor-suppressing properties of the garment are lost after only a few washings.

### SUMMARY OF THE INVENTION

The applicant has now identified a fully effective solution of the problems outlined above thanks to a method capable of binding an aluminium chloride based deodorizing agent to a textile fiber, without in any way impairing the quality of the latter and above all ensuring that the association between the agent and the fiber resists to quite a lot of washings, and thus that a garment can be obtained that maintains its odor-suppressing properties for a long time.

The essential features of the method according to the invention are defined in the first of the appended claims. The dependent claims specify advantageous embodiments of the method.

### DESCRIPTION OF PREFERRED EMBODIMENTS

The characteristics and advantages of the method and composition for obtaining odor-suppressing textile products and textile products, namely garments, thus obtained according to the present invention will be brought out more clearly by the following description of its embodiments, which is given purely by way of example and is not to be taken as limitative in any way.

According to the invention, a deodorizing composition is prepared, to be used for treating the textile product destined to be worn on a user's body, both a finished product (garment) and in nature of a raw or semifinished material (fabric, yarn etc.).

A simple example of a deodorizing composition for applying the deodorizing active principle (agent) to garments made of cotton consists of an aqueous bath prepared as follows:
4% in volume of a 20% aqueous solution of aluminium chloride;
5÷20 g/l of a polymeric binder, such as acrylic resin;
10÷20 g/l of a cationic surfactant, e.g. a common quaternary-ammonium-based fixative for dyes;
2÷5 g/l of a softener, e.g. a perfumed Henkel® softener.

The garments to be treated are immersed for a few minutes (no more than 20 minutes) in the bath prepared in this manner, which is brought to a temperature of about 90° C. When a fibrous material containing wool is involved, the polymeric binder will preferably be a silicon based resin, and the temperature of the bath around 50° C.

The treatment is completed with a phase of rinsing and drying with hot air at a temperature suitable for the polymerization of the resin (normally variable between 70° and 180°). The treatment time and temperature may however be varied according to the particular machine employed. The indications provided above are considered to be optimal in the case of treatment with, for example, a centrifugal washing machine.

In a different reduction to practice of the method according to the invention, the active principle can be applied prior to the actual manufacturing of the garments, i.e. to the fabrics from which these garments are to be made. This can be done, for example, in a continuous process by carrying out the impregnation in a foulard machine with a bath containing 5÷10 g/l of the active principle in emulsified form with a non-ionic surfactant, 3÷5 g/l of emulsified acrylic resin, in a pH made slightly acid by means of acetic acid (pH=5) in case of wool-based fabrics, or in a neutral pH in case of fabrics with a cellulose base. After wringing the fabric, it is dried in a “Rameuse” machine at the temperature which is necessary to assure the polymerization of the acrylic resin.

In the compositions suggested above the polymeric binder obviously has the fundamental function of fixing the active principle to the textile fiber, holding it and thus making the association resistant to subsequent washings of the textile product. The aluminium chloride is released very slowly, thus assuring its deodorizing action in the course of time. Neither the silicon resin nor the acrylic resin causes any appreciable alteration of the softness characteristics of the fiber and they are therefore particularly suitable for this purpose. Other types of resins may however be used—e. g. butadiene, polyurethane, polyamide or acrylonitrile based resins—even combined in appropriate proportions.

In case of materials with a woolen base it is preferable to use resins capable of being polymerized at low temperatures, like those with radical-type polymerization mechanisms. In any case, the softener may serve to attenuate a possible stiffening effect deriving from the presence of the resin.

Application to textile fibers, for example and typically in accordance with the modalities described above, represents a particularly advantageous reduction to practice of the invention, because it assures an optimal effectiveness of the deodorizing action without in any way altering either the appearance or the original softness of the supporting materials, which will also remain wholly free of smell.

The combination of the aluminium chloride with the polymeric binder results in a stable adhesion to the textile support, ensuring that the deodorizing agent is released persistently in the course of time, and this even after a number of washings. Garments treated in the manner just described, when worn, suppress the development of bad odors from the body, even after an intense and prolonged sweating. Experimental tests carried out on people particularly prone to this kind of problem, who for this purpose were made to wear garments treated according to the invention, demonstrated the complete disappearance of the bad smell. Moreover, repeated washings of the garments did not bring out any perceptible decay of the deodorizing properties. No allergic manifestation of any kind to the detriment of the wearers were revealed by any of the tests. It should also be noted that the deodorizing principle in question, apart from being inert, does not interact with the sebaceous secretion and is therefore absolutely tolerable from a hygienic and sanitary point of view.

Application to textile fibers, for example and typically in accordance with the modalities described above, assures an optimal effectiveness of the deodorizing action without in any way altering either the appearance or the original softness of the supporting materials, which will also remain wholly free of any particular smell. However, this application can be carried out with various modalities, especially in accordance with the variations of the material for which it is intended and therefore also of the machines that are employed. For example, the latter could include the so-called “Dutch machines”, where the movement of the bath is more gentle and thus avoids the physical alteration of materials made of wool fibers. More generally speaking, the aluminium chloride and the polymeric binder can also be applied via impregnation or spraying systems.

Variants and/or modifications can be brought to the method and composition for obtaining odor-suppressing textile products and textile products, namely garments, thus obtained without thereby departing from the scope of the invention itself as defined in the appended claims.

What is claimed is:

1. A method for obtaining an odor-suppressing textile product intended to be worn on the human body, the method making use of an active deodorizing ingredient that includes aluminum chloride or a substance capable of liberating aluminum chloride, wherein the product is partially or completely treated with a composition which comprises, in addition to the active ingredient, a polymeric binder for stably fixing the active ingredient to the product and releasing it gradually over time.

2. The method set forth in claim 1, wherein the polymeric binder comprises at least one resin selected from a group that includes: acrylic-based resin, silicone-based resin, butadiene-based resin, polyurethane-based resin, polyamide-based resin and acrylonitrile-based resin; the active ingredient and the resin being distributed in a composition with which the product is impregnated or sprayed.

3. The method set forth in claim 1, wherein the polymeric binder comprises at least one resin selected from a group that includes: acrylic-based resin, silicone-based resin, butadiene-based resin, polyure-thane-based resin, polyamide-based resin and acrylonitrile-based resin; the active ingredient and the resin being distributed in an aqueous bath in which the product is immersed.

4. The method set forth in claim 3, wherein the aqueous bath comprises about 4% in a volume of an approximately 20% aqueous solution of the active ingredient, the polymeric binder having a concentration between about 5 g/l and about 20 g/l, the aqueous bath further including a selected cationic surfactant and a softener having a concentration, respectively, between about 10 g/l and about 20 g/l and between about 2 g/l and about 5 g/l.

5. The method set forth in claim 4, wherein the aqueous bath is brought to a temperature of at least about 40° C.

6. The method set forth in claim 3, wherein the active ingredient has a concentration generally within a range of 5 g/l and 10 g/l and is emulsified with a selected non-ionic surfactant, the resin being an emulsified acrylic resin and having a concentration between about 3 g/l and about 5 g/l, the bath having a slightly acidic pH using acetic acid for wool-base products or a neutral pH with products having a cellulose base.

7. The method set forth in claim 6, wherein the textile product, subsequent to the bath, is wrung and dried with hot air at a temperature between about 70° C. and about 180° C.

8. A composition for partially or completely treating a textile product intended to be worn on the human body which utilizes an active deodorizing ingredient that includes aluminum chloride, or a substance capable of liberating aluminum chloride, which comprises, in addition to the active ingredient, a polymeric binder for stably fixing the active ingredient to the products and releasing it gradually over time.

9. The composition set forth in claim 8, wherein the polymeric binder comprises at least one resin selected from a group that includes: acrylic-based resin, silicone-based resin, butadiene-based resin, polyurethane-based resin, polyamide-based resin and acrylonitrile-based resin; the active ingredient and the resin being distributed in an aqueous bath in which the products are immersed.

10. The composition according to set forth in claim 8, which comprises about 4% in a volume of an approximately 20% aqueous solution of the active ingredient, the polymeric binder having a concentration between about 5 g/l and about 20 g/l, the aqueous bath further including a selected cationic surfactant and a softener having a concentration, respectively, between about 10 g/l and about 20 g/l and between about 2 g/l and about 5 g/l.

11. The composition set forth in claim 9, wherein the active ingredient has a concentration generally within a range of 5 g/l and 10 g/l and is emulsified with a selected non-ionic surfactant, the resin being an emulsified acrylic resin and having a concentration between about 3 g/l and about 5 g/l, the bath having a pH made slightly acid by acetic acid for wool-base products or a neutral pH for products with a cellulose base.

12. A textile product partially or totally impregnated or treated with a composition having a polvmeric binder which comprises at least one resin selected from a group that includes: acrylic-based resin silicone-based resin, butadiene-based resin, polyurethane-based resin, polyamide-based resin and acrylonitrile-based resin; the active ingredient and the resin being distributed in an aqueous bath in which the product is immersed.
